(19) United States
(12) Patent Application Publication
(10) Pub. No.: US 2007/0204865 A1
(43) Pub. Date: Sep. 6, 2007

(54) Aerosol generating apparatus and method, and film forming apparatus and method using the same

(75) Inventor: Takami Arakawa, Kaisei-machi (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation

(21) Appl. No.: 11/707,118

(22) Filed: Feb. 16, 2007

(30) Foreign Application Priority Data
Mar. 2, 2006 (JP) 2006-056316

(51) Int. Cl. A62B 7/00; A61M 15/00; A62B 9/00

(52) U.S. Cl. 128/203.15; 128/203.12; 128/200.24

(57) ABSTRACT

An aerosol generating apparatus capable of stably generating an aerosol with controlled concentration, and a film forming apparatus using the aerosol generating apparatus. The aerosol generating apparatus includes a powder containing chamber formed with an opening for leading out powder contained therein, an assist gas lead-in part for assisting lead-out of the powder when the powder is led out from the opening, and a dispersion gas lead-in part for supplying a gas for dispersing the powder led out from the opening. Further, the film forming apparatus includes the aerosol generating apparatus, a substrate stage for holding a substrate, and a nozzle for injecting the aerosol generated by the aerosol generating apparatus toward the substrate.

### BACKGROUND OF THE INVENTION

1. Field of the Invention

The present invention relates to apparatus and method for generating an aerosol by dispersing raw material powder in a gas, and film forming apparatus and method for performing film formation by using the aerosol.

2. Description of a Related Art

In recent years, with the developments of MEMS (micro electro mechanical systems) related devices, elements such as multilayered ceramic capacitors and piezoelectric actuators have been microfabricated still further and packaged more densely. Accordingly, the manufacture of such elements by using film forming technologies has been actively studied.

Recently, an aerosol deposition (AD) method as one of the film forming technologies utilizing the collision and deposition phenomenon of solid particles has received attention. The AD method is a film forming method of injecting an aerosol generated by dispersing fine powder of a raw material in a gas from a nozzle toward a substrate and allowing the powder to collide with the substrate or the previously formed film to deposit the raw material on the substrate. Here, the aerosol refers to “a colloid system including a dispersion phase consisting of solid or liquid particles and a dispersion medium consisting of a gas” (“Basis of Aerosol Studies” written by Kanji Takahashi, Morikita Publishing, 1st edition, p. 1). According to the AD method, dense and strong films with low porosity can be formed, and therefore, the performance of the above-mentioned microelements may be improved.

In order to form a good-quality film having homogeneous thickness and density by using the AD method, it is important to continue to supply an aerosol with homogeneous concentration over a long period. For the purpose, a specific amount of powder should be continuously supplied to an aerosol generation mechanism for dispersing the powder in a gas.

As a related technology, Japanese Patent Application Publication JP-A-5-186864 discloses a powder supply apparatus for use in so-called plasma vapor deposition of depositing powder on a sample by supplying the powder with a carrier gas into plasma. The powder supply apparatus has a powder supply unit for supplying powder that has been agitated therein and a distributor for dispersing and returning aggregated fine powder of the powder supplied from the powder supply unit into original fine powder.

Further, Japanese Patent Application Publication JP-A-5-239625 discloses a powder supply apparatus for use in plasma vapor deposition and including a rotating disk having a groove cut on a circle in a predetermined position located from a center thereof and a powder container arranged above the rotating disk and for dropping powder into the groove of the rotating disk. In the powder supply apparatus, rotational agitating blades are attached to the bottom part of the powder container and the agitating blades are shaped such that the powder may move around the agitating blades.

Furthermore, Japanese Patent Application Publication JP-P2005-68542A discloses a powder supply apparatus for use in plasma vapor deposition and including a powder containing chamber with rotatable agitating blades and a powder drop hole, a rotating disk formed with a groove on a circle facing the powder drop hole, a powder drawing pipe having a tip inserted into a location within the groove adjacent to a location of the groove into which the powder drops, a hole for passing a carrier gas to be sprayed to one of the locations in the groove.

Further, Japanese Patent Application Publication JP-P2003-275631A discloses an aerosol generating apparatus including a powder containing unit, powder transporting means, and aerosolizing means. In the aerosol generating apparatus, the powder transporting means is circulation type transporting means formed with a groove to be filled with powder from the powder containing unit, the aerosolizing means has a gas lead-in port for spraying a gas to a part of the groove and an aerosol lead-out port nearby. A carrier gas introduced from the gas lead-in port is sprayed to a part of the powder transported by the powder transporting means and aerosolized, and then, the aerosol is led out from the aerosol lead-out port. JP-P2003-275631A also discloses a composite structure fabricating apparatus including such an aerosol generating apparatus and a nozzle for spraying the aerosol to a base material.

Furthermore, Japanese Patent Application Publication JP-P2005-113261A discloses an aerosol generating apparatus for use in a composite structure fabricating apparatus for forming a structure consisting of fine powder constituent materials on a base material by injecting an aerosol formed by dispersing fine particles of a brittle material from a nozzle toward the base material and allowing the aerosol to collide with a surface of the base material. The aerosol generating apparatus includes a powder containing unit capable of replenishing fine particles, a powder supply unit for continuously carrying a specific amount of fine particles from the powder containing unit, and an aerosolizing unit for aerosolizing the fine particles from the powder supply unit with a gas from a gas lead-in unit and leading out the aerosol from a lead-out unit. JP-P2005-113261A also discloses a composite structure fabricating apparatus including such an aerosol generating apparatus and a nozzle for spraying the aerosol to the base material.

Here, according to the powder supply apparatuses disclosed in JP-A-5-186864 and JP-A-5-239625, by using a powder supply disk on which a groove with specified width and depth is formed, a specified amount of powder according to the volume of the groove can be supplied. Accordingly, it is possible to generate an aerosol with stable concentration by dispersing the powder supplied from the powder supply disk with a gas. However, when it is desired that an aerosol with low concentration of powder is generated by increasing the amount of the dispersion gas, a large amount of gas flows in the powder supply disk and the powder is scattered by the gas, and therefore, the control of the aerosol concentration becomes difficult.

In this regard, in JP-P2005-68542A, JP-P2003-275631A and JP-P2005-113261A, variations in the amount of the powder after carried are suppressed by generating an aerosol by directly spraying a carrier gas to the groove of the powder supply disk (rotating disk). However, in the powder supply apparatuses disclosed in these documents, the powder is placed in the groove of the powder supply disk by naturally dropping the powder from the opening of the powder containing chamber. Accordingly, when the powder fluidity is low, the powder is hard to drop from the opening, and thus, no powder may be supplied or control of the amount of powder can not be taken. Consequently, the control of the aerosol concentration becomes difficult.

Furthermore, in JP-A-5-186864, an agitator and agitating blades are provided for agitating the powder placed within an airtight container. A gap enough for preventing powder from being stuck is provided between the agitating blades and the groove of the powder supply disk. Further, the agitator and the powder supply disk are positioned such that there is no mechanical overlap between them in order to prevent occurrence of contamination (impurities) due to the meshing with each other. Accordingly, when the powder fluidity is low, the powder may not move into the groove. Further, when the gap is too large, the amount of powder placed in the groove may be instable.

### SUMMARY OF THE INVENTION

Therefore, in view of the above-mentioned problems, a first purpose of the present invention is to provide aerosol generating apparatus and method capable of stably generating an aerosol with controlled concentration. Further, a second purpose of the present invention is to provide film forming apparatus and method for performing film formation by using the aerosol generated by the aerosol generating apparatus or method.

In order to achieve the above-mentioned purposes, an aerosol generating apparatus according to one aspect of the present invention includes: a powder containing chamber formed with an opening for leading out powder contained therein; assisting means for assisting lead-out of the powder when the powder is led out from the opening; and gas supplying means for supplying a gas for dispersing the powder led out from the opening.

Further, a film forming apparatus according to one aspect of the present invention is a film forming apparatus using a film forming method of depositing raw material on a substrate by spraying an aerosol, in which powder of the raw material is dispersed in a gas, toward the substrate, and the apparatus includes: (i) an aerosol generating apparatus including a powder containing chamber formed with an opening for leading out powder contained therein, assisting means for assisting lead-out of the powder when the powder is led out from the opening, and gas supplying means for supplying a gas for dispersing the powder led out from the opening; (ii) a substrate stage for holding a substrate; and (iii) a nozzle for injecting the aerosol generated by the aerosol generating apparatus toward the substrate.

An aerosol generating method according to one aspect of the present invention includes the steps of: (a) leading out powder contained in a powder containing chamber formed with an opening from the opening while assisting lead-out of the powder; and (b) dispersing the powder led out from the opening with a gas.

Further, a film forming method according to one aspect of the present invention includes the steps of: (a) leading out powder of a raw material contained in a powder containing chamber formed with an opening from the opening while assisting lead-out of the powder; (b) generating an aerosol by dispersing the powder led out from the opening with a gas; and (c) depositing raw material powder on a substrate by spraying the aerosol generated at step (b) from a nozzle toward the substrate to allow the powder to collide with the substrate.

According to the present invention, when the powder is led out from the opening formed in the powder containing chamber, the lead-out is assisted by the assisting means, and thus, the powder can be led out from the opening more smoothly. Thereby, the supplied amount of powder can be stabilized and the supplied amount of powder can be easily adjusted, and therefore, the aerosol with controlled concentration can be generated for a long period. Accordingly, by using thus generated aerosol, a film with stable film quality including film thickness and density can be formed and the control of film thickness and density can be taken more easily.

### BRIEF DESCRIPTION OF THE DRAWINGS

FIGS. 1A and 1B are diagrams for explanation of an aerosol generating apparatus according to the first embodiment of the present invention;

FIG. 2 is an enlarged sectional view of a part around the top of a rotating disk shown in FIGS. 1A and 1B;

FIG. 3 is an enlarged sectional view showing a part near an aerosol lead-out part shown in FIGS. 1A and 1B;

FIG. 4 is a sectional view showing a configuration of an aerosol generating apparatus according to the second embodiment of the present invention;

FIG. 5 is a sectional view showing a part of the aerosol generating apparatus according to the third embodiment of the present invention;

FIG. 6 is a sectional view showing a configuration of an aerosol generating apparatus according to the fourth embodiment of the present invention;

FIG. 7 is a sectional view showing a part of the aerosol generating apparatus according to the fifth embodiment of the present invention;

FIG. 8 is a sectional view showing a part of the aerosol generating apparatus according to the sixth embodiment of the present invention;

FIGS. 9A and 9B are diagrams for explanation of an aerosol generating apparatus according to the seventh embodiment of the present invention;

FIG. 10 is a diagram for explanation of an aerosol generating apparatus according to the eighth embodiment of the present invention;

FIG. 11 is a schematic diagram showing a configuration of a film forming apparatus according to one embodiment of the present invention;

FIG. 12 shows conditions in Experiment 1;

FIG. 13 shows results in Experiment 1;

FIG. 14 shows conditions in Experiment 2;

FIG. 15 shows results in Experiment 2;

FIG. 16 shows results in Experiment 2; and

FIG. 17 shows results in Experiment 3.

### DESCRIPTION OF THE PREFERRED EMBODIMENTS

Hereinafter, preferred embodiments of the present invention will be explained in detail by referring to the drawings. The same component elements are assigned with the same reference numerals and the description thereof will be omitted.

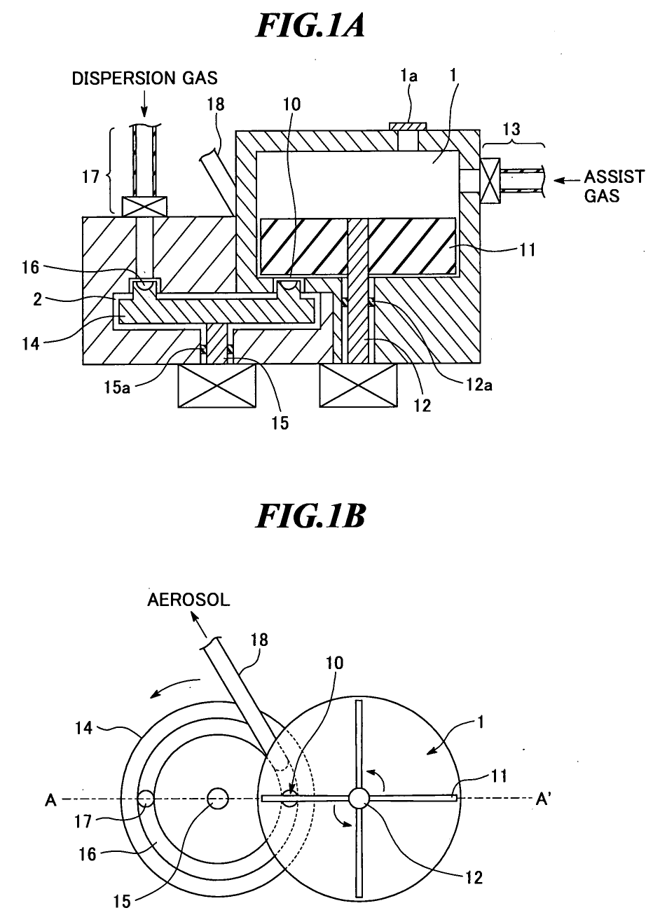

FIG. 1A is a sectional view showing a configuration of an aerosol generating apparatus according to the first embodiment of the present invention. Further, FIG. 1B is a top view showing the interior of the aerosol generating apparatus according to the embodiment. FIG. 1A shows a section along dashed-dotted line A-A′ in FIG. 1B.

As shown in FIGS. 1A and 1B, the aerosol generating apparatus includes a powder containing chamber 1 and an aerosol generating chamber 2.

The powder containing chamber 1 is a chamber for containing powder, and a powder supply port 1a is provided in an upper base thereof and an opening 10 is formed in a lower base. The powder containing chamber 1 and the aerosol generating chamber 2 are connected via the opening 10.

The powder containing chamber 1 includes agitating blades 11 to be driven by a motor for rotation. An O-ring 12a is fitted with a rotating shaft 12 of the agitating blades 11 and thereby airtightness within the powder containing chamber 1 is ensured. Although four agitating blades 11 are shown in FIG. 1B, the number of agitating blades may be appropriately varied. As a material of the agitating blades 11, a hard material such as a metal or a material advantageous in flexibility such as rubber, silicon rubber, and Teflon® may be used. Alternatively, a combination of those materials such that metal blades are covered with rubber may be used.

Powder is placed in the powder containing chamber 1 and the powder is agitated with the agitating blades 11. Thereby, the powder drops from the opening 10 and is led out to the aerosol generating chamber 2.

Further, an assist gas lead-in part 13 is provided for assisting or promoting the lead-out action of the powder from the opening 10. The assist gas lead-in part 13 includes a pipe and a valve and, for example, a compressed gas cylinder is connected to the opposite end of the pipe. The same kind of assist gas as that of a dispersion gas, which will be described later, is desirably used.

A rotating disk 14 to be driven by a motor for rotation is provided in the aerosol generating chamber 2. An O-ring 15a is fitted with a rotating shaft 15 of the rotating disk 14 and thereby airtightness within the aerosol generating chamber 2 is ensured.

A groove 16 having predetermined width and depth is formed along the circumference of the rotating disk 14. The rotating disk 14 is positioned such that the groove 16 faces the opening 10 of the powder containing chamber 1. The rotating disk 14 rotates while receiving the powder dropping from the opening 10 with the groove 16, and thereby, carries the powder at a fixed rate. Although the section of the groove 16 is in a semi-circular shape in FIG. 1A, it may be in other shapes than semi-circular, for example, a rectangular shape or V-shape.

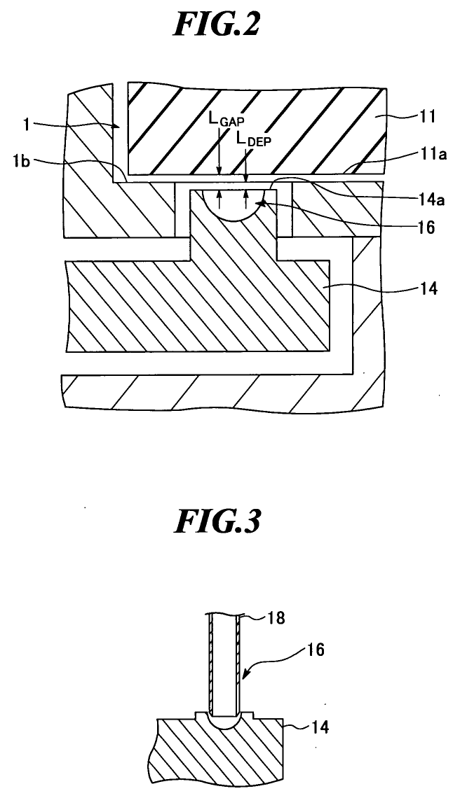

FIG. 2 is an enlarged view for explanation of a positional relationship among the bottom part of the powder containing chamber 1, the agitating blades 11, and the rotating disk 14 shown in FIGS. 1A and 1B. In FIG. 2, length L_{GAP} represents a gap between the lowermost part 11a of the agitating blades 11 and the top (the uppermost part of the wall of the groove 16) 14a of the rotating disk 14, and length L_{DEP} represents the depth of the top 14a of the rotating disk 14 seen from the inner bottom surface 1b of the powder containing chamber 1. If the length L_{DEP} is positive, the position of the top 14a of the rotating disk 14 is lower than the inner bottom surface 1b of the powder containing chamber 1.

As shown in FIG. 2, space is provided between the agitating blades 11 and the inner wall and inner bottom surface of the powder containing chamber 1. This is for preventing the mixture of contamination in the powder because the agitating blades 11 and the inner wall and inner bottom surface of the powder containing chamber 1 rub against and scrape out each other to produce contamination. Such space is necessary when the peripheral part of the agitating blades 11 is formed of a hard material such as a metal, however, not necessarily provided when the part is formed of a soft material such as rubber.

Further, space is also provided between the agitating blades 11 and the top 14a of the rotating disk 14 (i.e., the length L_{GAP}>0). This is for preventing the production of contamination by the rubbing between the agitating blades 11 and the rotating disk 14. Similarly, such space is necessary when the peripheral part of the agitating blades 11 is formed of a hard material such as a metal, however, not necessarily provided when the part is formed of a soft material such as rubber. In FIG. 2, the top 14a of the rotating disk 14 is positioned slightly lower than the inner bottom surface 1b of the powder containing chamber 1, and therefore, space is naturally formed between the rotating disk 14 and the agitating blades 11. However, the position of the top 14a of the rotating disk may be aligned with the inner bottom surface 1b of the powder containing chamber 1 (i.e., L_{DEP}=0), or higher than the inner bottom surface 1b (i.e., L_{DEP}<0). Note that, in order to efficiently fill the groove 16 with the powder, it is desirable that the position of the top 14a is level with or lower than the inner bottom surface 1b of the powder containing chamber 1 (i.e., L_{DEP}≧0).

In the embodiment, the distance L_{GAP} between the agitating blades 11 and the top 14a of the rotating disk is set to 1 mm or less, preferably 0.5 mm or less, and more preferably as small as possible. This is because, if the distance between them is too long, even when the powder drops within the opening 10, it becomes difficult to reliably fill the groove 16 with the powder, and thereby, the amount of the powder to be carried largely varies. Therefore, when the peripheral part of the agitating blades 11 is formed of a soft material such as rubber, the distance may be set as L_{GAP}=0. Further, in order to minimize the variations in the amount of the powder, it is desirable that the distance L_{GAP} is maintained with high accuracy even during driving of the aerosol generating apparatus.

Referring to FIGS. 1A and 1B again, a dispersion gas lead-in part 17 and an aerosol lead-out part 18 are provided in the aerosol generating chamber 2.

The dispersion gas lead-in part 17 includes a pipe and a valve and, for example, a compressed gas cylinder is connected to the opposite end of the pipe. As a dispersion gas, nitrogen (N₂), oxygen (O₂), helium (He), argon (Ar), a mixed gas thereof, dry air, or the like is used. As shown in FIG. 1A, an outlet of the dispersion gas introduced by the dispersion gas lead-in part 17 into the aerosol generating chamber 2 is provided to face the groove 16 of the rotating disk 14.

FIG. 3 is an enlarged sectional view showing the part near the aerosol lead-out part 18 shown in FIG. 1B. As shown in FIG. 3, the aerosol lead-out part 18 is a tube (tubular body) with an opening of the leading end positioned to face the groove 16. The aerosol lead-out part 18 may be directed perpendicularly to the groove 16 as shown in FIG. 3, or tilted toward right and left directions or depth direction of the drawing. Further, in order to reliably guide the generated aerosol to the aerosol lead-out part 18, it is desirable that the leading end of the aerosol lead-out part 18 is positioned inside of the groove 16. The position of the aerosol lead-out part 18 may be any location on the circle of the groove 16, and it is more desirable that the position is located not so far from the position immediately below the opening 10 of the powder containing chamber 1. This is because the distance of the powder to be carried becomes shorter and the powder hardly attaches to the inner wall (specifically, the ceiling) of the aerosol generating chamber 2. The opposite end of the aerosol lead-out part 18 is connected to a pipe formed of a flexible material, for example.

In the aerosol generating apparatus, desired powder is placed in the powder containing chamber 1 and the agitating blades 11 are driven, and the rotating disk 14 is rotated in the aerosol generating chamber 2 and the dispersion gas is started to be sprayed to the groove 16 of the rotating disk 14.

The powder contained in the powder containing chamber 1 drops into the groove 16 through the opening 10 while being agitated by the agitating blades 11. Simultaneously, the assist gas is introduced into the powder containing chamber 1 to form airflow within the opening 10. The assist gas may be continuously introduced or intermittently introduced. The airflow acts as a driving force for assisting or promoting the lead-out of the powder. Thereby, the powder drops from the opening 10 into the groove 16 more smoothly. The powder that has dropped into the groove 16 is deposited and carried according to the rotational speed of the rotating disk 14.

On the other hand, in the groove 16 of the rotating disk 14, the dispersion gas sprayed thereto flows along the groove 16 and forms airflow. The dispersion gas flows from the opening of the leading end of the aerosol lead-out part 18 thereinto. Simultaneously, a suction force toward the inside of the aerosol lead-out part 18 is produced around the aerosol lead-out part 18. By the suction force, the powder deposited on the groove 16 flows together with the dispersion gas into the aerosol lead-out port 18. Thus generated aerosol is introduced into a film forming apparatus or the like via a flexible pipe connected to the aerosol lead-out port 18 or the like.

As described above, according to the embodiment, the powder drops from the opening 10 more smoothly by introducing the assist gas into the powder containing chamber 1, and therefore, the amount of the powder to be deposited on the groove 16 can be stabilized. Further, according to the embodiment, since the gap between the agitating blades 11 and the top 14a of the rotating disk 14 is set to 1 mm or less, the amount of the powder to be packed in the groove 16 can be stabilized while the production of contamination is suppressed. Consequently, by changing the rotational speed of the rotating table 14, the amount of the powder to be supplied per unit time can be accurately controlled, and therefore, the aerosol with stable concentration can be generated for a long period and the aerosol concentration can be easily controlled.

In the embodiment, both the assist gas lead-in part 13 and the agitating blades 11 at the specified distance from the rotating disk 14 are provided as means for assisting or promoting the lead-out of the powder, however, even when only one of them is provided, an aerosol can be generated more stably compared to the conventional apparatus.

As a modified example of the aerosol generating apparatus according to the first embodiment of the present invention, in the case where the diameter of the opening 10 of the powder containing chamber 1 is made smaller, the assist gas lead-in part 13 may be used as a pressure regulating part of the powder containing chamber 1. In the case where the diameter of the opening 10 is small, introduction of the assist gas forms a pressurized condition within the powder containing chamber 1. Therefore, when the pressure within the powder containing chamber 1 is maintained higher than that of the aerosol generating chamber 2, the pressure difference acts as a driving force for assisting or promoting the lead-out of the powder, and thus, the powder is dropped from the opening 10 into the groove 16 more smoothly.

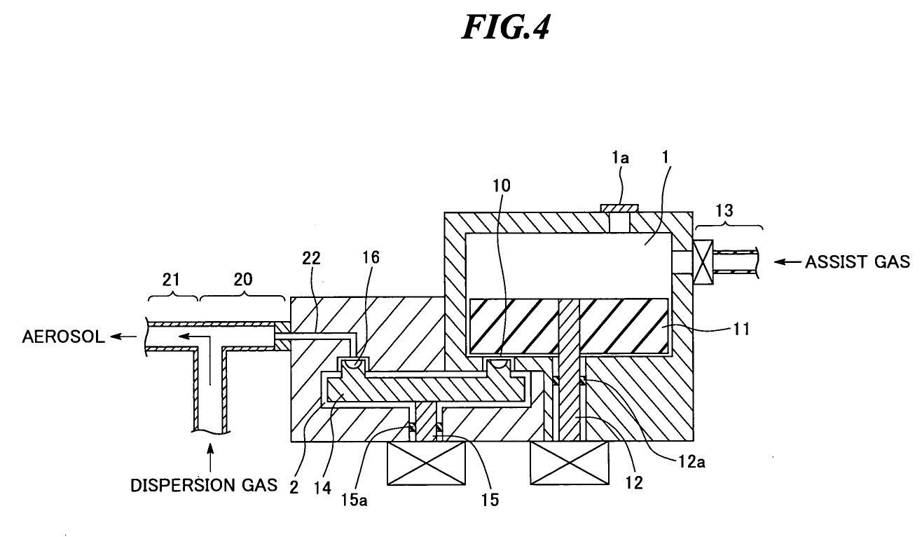

Next, an aerosol generating apparatus according to the second embodiment of the present invention will be explained with reference to FIG. 4. Although the dispersion gas is directly sprayed to the groove 16 of the rotating disk 14 in the aerosol generating apparatus shown in FIGS. 1A and 1B, the way of spraying is not necessarily adopted here.

The aerosol generating apparatus shown in FIG. 4 has a dispersion gas lead-in part 20 and an aerosol lead-out part 21 in place of the dispersion gas lead-in part 17 and the aerosol lead-out part 18 shown in FIG. 1A. Further, a powder lead-out path 22 for connecting a part of the groove 16 of the rotating disk 14 and the dispersion gas lead-in part 20 is formed in the aerosol generating apparatus. The powder lead-out path 22 has a diameter that differs depending on the diameter and fluidity of the powder to be supplied or the like, and is a narrow tube of about 1 mm to 3 mm, for example.

When the powder is placed in the powder containing chamber 1 of the aerosol generating apparatus and the operation is started, the powder drops from the opening 10 by the assist of the assist gas and is deposited on the groove 16. The powder is carried to the position of the powder lead-out path 22 by the rotation of the rotating disk 14. Then, the powder placed in the groove 16 passes through the powder lead-out path 22 together with the assist gas at a high speed, and is injected to the dispersion gas lead-in part 20. Simultaneously, the powder is extremely highly dispersed due to the expansion force generated by the difference between the diameter of the powder lead-out path 22 and the dispersion gas lead-in part 20. Furthermore, the dispersion gas passes through there and the powder is led out together with the dispersion gas. Thus, an aerosol is generated.

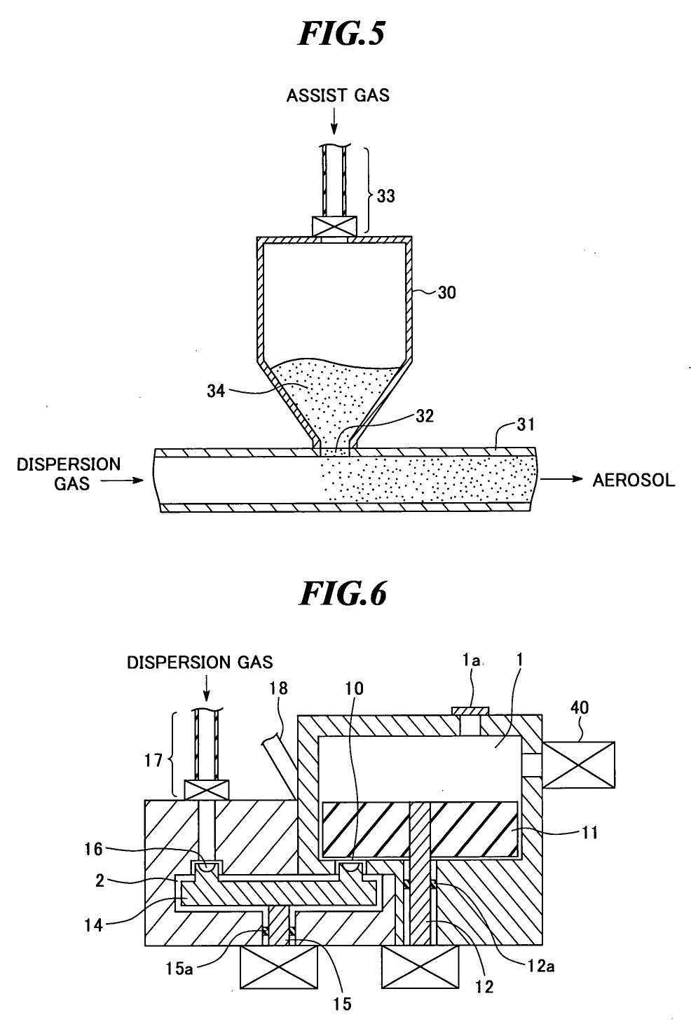

Next, an aerosol generating apparatus according to the third embodiment of the present invention will be explained. FIG. 5 is a sectional view showing part of the aerosol generating apparatus according to the embodiment.

As shown in FIG. 5, the aerosol generating apparatus includes a powder containing chamber 30 and a dispersion gas supply pipe 31.

The powder containing chamber 30 has a container having a funnel shape or a combination of a funnel shape and a cylindrical shape. As the container, for example, a hopper for allowing powder to drop downwardly is known. Further, the dispersion gas supply pipe 31 is a tubular body for carrying the dispersion gas supplied from the compressed gas cylinder or the like, and an opening 32 is formed in part thereof. The respective parts are arranged such that the position of the opening 32 and the opening formed at the leading end (the funnel part) of the powder containing chamber 30 are aligned.

Furthermore, an assist gas lead-in part 33 is provided in the powder containing chamber 30 for assisting or promoting the lead-out action of the powder placed within to the assist gas lead-in part 33. The assist gas lead-in part 33 includes a pipe and a valve and, for example, a compressed gas cylinder is connected to the opposite end of the pipe.

When powder 34 is placed in the powder containing chamber 30, the powder moves along the slope of a circular cone and is guided to the opening, and drops into the dispersion gas supply pipe 31. Simultaneously, the assist gas is introduced into the powder containing chamber 1 to form airflow within the powder containing chamber 30. The assist gas may be continuously introduced or intermittently introduced. The airflow acts as a driving force for assisting or promoting the lead-out of the powder. Thereby, the powder passes through the opening more smoothly and drops into the dispersion gas supply pipe 31. The powder is dispersed with the dispersion gas within the dispersion gas supply pipe 31, and therefore, an aerosol is generated.

Thus, according to the embodiment, by introducing the assist gas into the powder containing chamber that has a slope in part of the inner wall thereof, the powder can be stably supplied to the aerosol generating part by a simple configuration.

As a modified example of the aerosol generating apparatus according to the third embodiment of the present invention, in the case where the diameter of the opening of the powder containing chamber 30 is made smaller, the assist gas lead-in part 33 may be used as a pressure regulating part of the powder containing chamber 30. In the case where the diameter of the opening is small, introduction of the assist gas forms a pressurized condition within the powder containing chamber 30. Therefore, when the pressure within the powder containing chamber 30 is maintained higher than that within the dispersion gas supply pipe 31, the pressure difference acts as a driving force for assisting or promoting the lead-out of the powder, and thus, the powder can be supplied into the dispersion gas supply pipe 31 more smoothly.

Next, an aerosol generating apparatus according to the fourth embodiment of the present invention will be explained. FIG. 6 is a sectional view showing a configuration of the aerosol generating apparatus according to the embodiment.

As shown in FIG. 6, the aerosol generating apparatus includes a knocker 40 in place of the assist gas lead-in part 13 shown in FIG. 1A. Other constitution is the same as that shown in FIGS. 1A and 1B.

Here, the knocker is a device mounted on a container for containing powder, a pipe for carrying powder, or the like for removing the attachment and clogging of the powder by providing vibration or impact to the container or the like. As the knocker, an air knocker for driving a piston by a force of compressed air and an electronic knocker for driving a hammer by electric power are known.

By driving the knocker 40 to provide vibration or impact to the powder containing chamber 1, the powder contained in the powder containing chamber 1 can be dropped from the opening 10 into the groove 16.

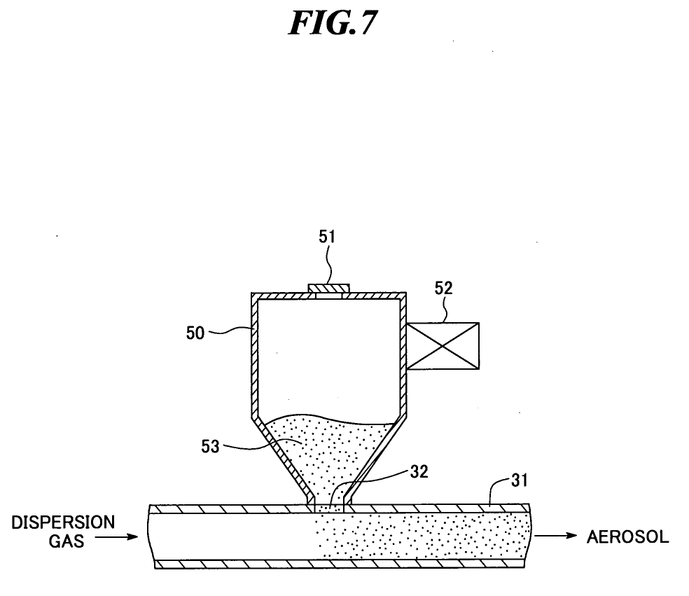

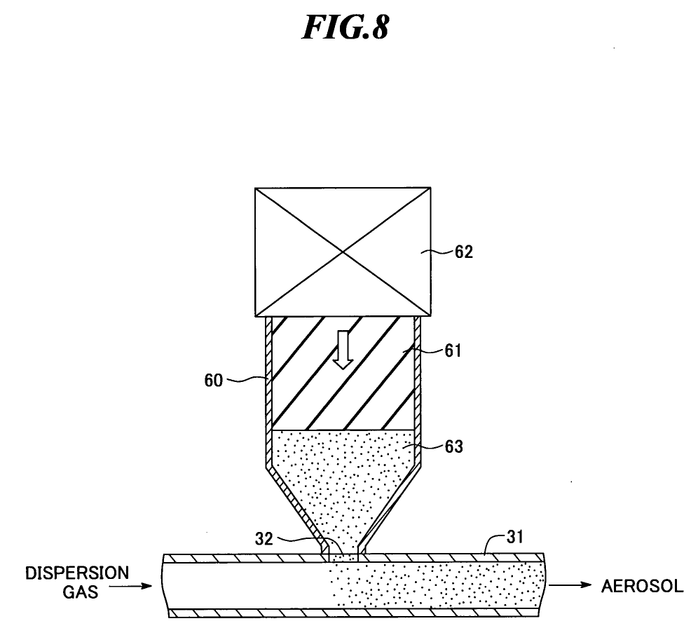

Next, an aerosol generating apparatus according to the fifth embodiment of the present invention will be explained. FIG. 7 is a sectional view showing part of the aerosol generating apparatus according to the embodiment.

As shown in FIG. 7, the aerosol generating apparatus includes a powder containing chamber 50 in place of the powder containing chamber 30 shown in FIG. 5. Other constitution is the same as that shown in FIG. 5.

The powder containing chamber 50 has a container having a funnel shape or a combination of a funnel shape and a cylindrical shape, and a powder supply port 51 is provided in the upper part thereof. Further, the respective parts are arranged such that the positions of the opening formed at the leading end of the funnel and the opening 32 of the dispersion gas supply pipe 31 are aligned. Furthermore, a knocker 52 is provided outside of the powder containing chamber 50.

When powder 53 is placed in the powder containing chamber 50, the powder moves along the slope of a circular cone and is guided to the opening. Simultaneously, the knocker 52 is driven to provide vibration or impact to the powder containing chamber 50, and thereby, the powder 53 can be allowed to drop into the dispersion gas supply pipe 31 more smoothly.

Next, an aerosol generating apparatus according to the sixth embodiment of the present invention will be explained. FIG. 8 is a sectional view showing part of the aerosol generating apparatus according to the embodiment.

As shown in FIG. 8, the aerosol generating apparatus has a powder containing chamber 60 in place of the powder containing chamber 30 shown in FIG. 5. Other constitution is the same as that shown in FIG. 5.

The powder containing chamber 60 has a container having a funnel shape or a combination of a funnel shape and a cylindrical shape. The respective parts are arranged such that the positions of an opening formed at the leading end of the funnel and the opening 32 of the dispersion gas supply pipe 31 are aligned. Further, a piston 61 is provided inside of the powder containing chamber 60. The piston 61 is driven by a driving device 62 such as a hydraulic cylinder, an air cylinder, and an electronic cylinder, for example.

When powder 63 is placed in the powder containing chamber 60, the powder moves along the slope of a circular cone and is guided to the opening. Simultaneously, the piston 61 is driven to push out the power 63 from the opening, and thereby, the powder 63 can be allowed to drop into the dispersion gas supply pipe 31 more smoothly.

Next, an aerosol generating apparatus according to the seventh embodiment of the present invention will be explained.

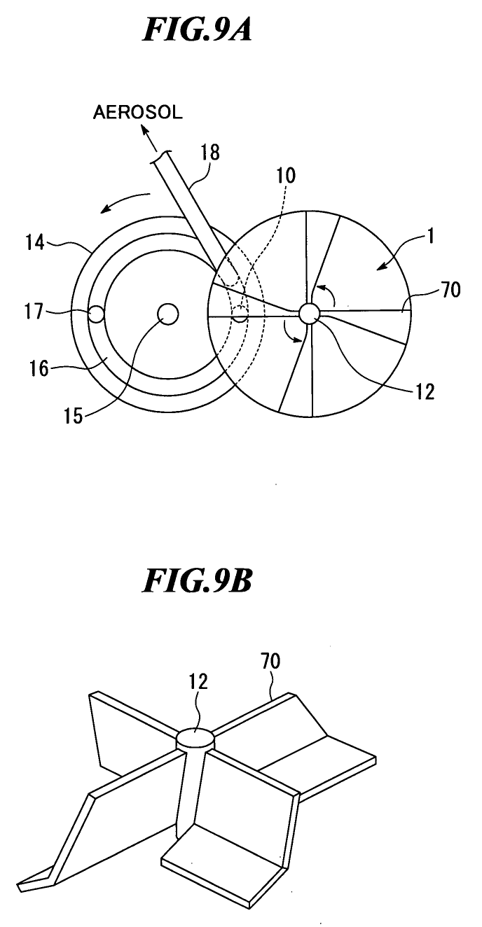

FIG. 9A is a top view showing the interior of the aerosol generating apparatus according to the embodiment. As shown in FIG. 9A, the aerosol generating apparatus has agitating blades 70 in place of the agitating blades 11 shown in FIG. 1B. Other constitution is the same as that shown in FIGS. 1A and 1B. Although four agitating blades 70 are shown in FIGS. 9A and 9B, the number of agitating blades may be appropriately varied.

The agitating blades 70 are positioned such that ends thereof are in contact with the bottom surface of the powder containing chamber 1. Desirably, the agitating blades 70 may be in contact with the side surface of the powder containing chamber 1.

FIG. 9B is a perspective view showing the agitating blades 70 shown in FIG. 9A. The respective agitating blades 70 are bent in the middle such that an angle formed by the end portion thereof and the bottom surface of the powder containing chamber 1 is small. Further, the agitating blades 70 are formed of a material advantageous in flexibility such as rubber, silicon rubber, and Teflon®, for example.

When the agitating blades 70 are rotated, the agitating blades 70 agitate the powder contained in the powder containing chamber 1 and the end portions thereof act as scrapers to press the powder entering the downside against the bottom surface. The pressing force acts as a driving force for assisting or promoting the lead-out of the powder. Thereby, the powder drops into the groove 16 more smoothly. Further, when the agitating blades 70 are formed of the above-mentioned material, the contact parts of the end portions of the agitating blades 70 with the bottom surface and side surface of the powder containing chamber 1 are no longer scraped out, and thus, mixture of the contamination (impurities) in the powder hardly occurs.

Here, the shape of the agitating blades is not limited to that shown in FIG. 7 as long as the agitating blades may be brought into contact with the bottom surface of the powder containing chamber at a relatively small angle. For example, the blades may be gently curved. Alternatively, the agitating blades may be attached to the rotating shaft at a slant. Furthermore, although the agitating blades (pressing scrapers) are rotated in the embodiment, they may be driven for parallel shift.

In the embodiment, it is also desirable that the gap between the lowermost part of the agitating blades 70 and the rotating disk 14 is set to 1 mm or less (i.e., L_{GAP}=L_{DEP}≦1 mm).

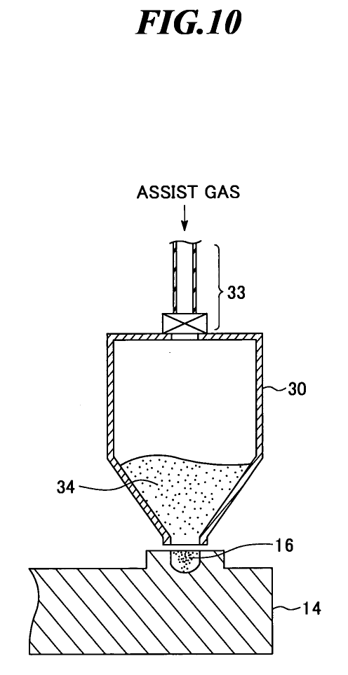

Next, an aerosol generating apparatus according to the eighth embodiment of the present invention will be explained. FIG. 10 is a sectional view showing part of the aerosol generating apparatus according to the embodiment.

The aerosol generating apparatus according to the embodiment is configured by providing the powder containing chamber 30 shown in FIG. 5 in place of the powder containing chamber 1 in the aerosol generating apparatus shown in FIGS. 1A and 1B. Other constitution is the same as that shown in FIGS. 1A and 1B.

In the embodiment, the powder that has been dropped with the assist by the assist gas is received by the groove 16 of the rotating disk 14. Then, the rotating disk 14 is rotated to carry the powder to a predetermined position, and the dispersion gas is introduced from the position to disperse the powder (see FIGS. 1A and 1B).

According to the embodiment, by varying the rotational speed of the rotating disk 14, the amount of the powder to be carried to the introduction location of the dispersion gas per unit time can be accurately controlled.

As a modified example of the aerosol generating apparatus according to the embodiment, the powder containing chamber 50 shown in FIG. 7 or the powder containing chamber 60 shown in FIG. 8 may be provided in place of the powder containing chamber 30.

As described above, the aerosol generating apparatuses according to the first to eighth embodiments of the present invention have been explained. A plurality of the assist means (the means for assisting or promoting the lead-out action of the powder) used in those embodiments may be combined. For example, the knocker 40 shown in FIG. 6 may be added to the aerosol generating apparatus shown in FIGS. 1A and 1B, or the agitating blades 70 shown in FIGS. 9A and 9B may be attached to the aerosol generating apparatus shown in FIG. 6.

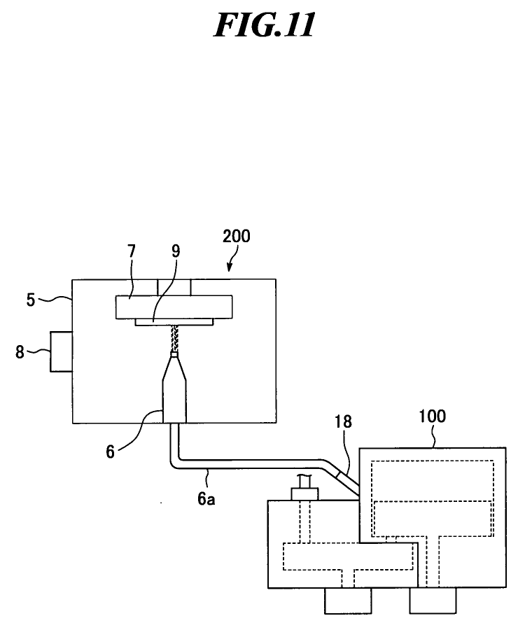

Next, a film forming apparatus according to one embodiment of the present invention will be explained. FIG. 11 is a schematic diagram showing a configuration of the film forming apparatus according to the embodiment.

As shown in FIG. 11, the film forming apparatus has an aerosol generating apparatus 100 and a film forming unit 200. One of the aerosol generating apparatuses according to the first to eighth embodiments of the present invention is applied to the aerosol generating apparatus 100.

The film forming unit 200 includes a film forming chamber 5, an injection nozzle 6, a substrate stage 7, and a vacuum pump 8. The interior of the film forming chamber 5 is maintained at a predetermined degree of vacuum by the vacuum pump 8. Further, the injection nozzle 6 injects an aerosol generated by the aerosol generating apparatus 100 and supplied via an aerosol carrier pipe 6a. The substrate stage 7 is a movable stage capable of three-dimensional movement and holds a substrate 9 on which a film is to be formed. The relative position and the relative speed between the substrate 9 and the injection nozzle 6 are adjusted by controlling the movement of the substrate stage 7.

In the film forming apparatus, desired raw material powder (e.g., metal powder or ceramic powder) is placed in the aerosol generating apparatus 100 and the substrate 9 is set on the substrate stage of the film forming unit 200. The diameter of raw material powder varies depending on the kind of film forming material, the relationship with a substrate material, and so on. For example, when a ceramic film of PZT (Pb (lead) zirconate titanate) is formed, the particle diameter is preferably set to about 0.1 μm to 10 μm. Further, as the substrate, various members of SUS (stainless steel), YSZ (yttria-stabilized zirconia), silicon, or the like may be used. Furthermore, a substrate with a metal film as an under layer or electrode layer formed on a substrate material may be used as the substrate 9.

When the aerosol generating apparatus 100 is driven, an aerosol in which the raw material powder is dispersed in a gas is generated. The aerosol is supplied to the injection nozzle 6 via the aerosol carrier pipe 6a. Then, the aerosol is injected toward the substrate 9 by the injection nozzle 6, and the raw material powder collides with the substrate 9, is crushed, adheres to the substrate due to mechanochemical phenomenon, and is deposited thereon. In this regard, the thickness of the film to be deposited on the substrate 9 can be controlled by adjusting the relative speed between the substrate 9 and the injection nozzle 6 and the number of reciprocation of the substrate.

As described above, according to the film forming apparatus according to the one embodiment of the present invention, a low-porosity and dense metal film, ceramic film, and so on can be formed using the AD method. Especially, when the ceramic film is formed, a high-hardness (strong) film can be formed. In the AD method, depending on the relationship between the hardness of the substrate material and the foundation layer material and the hardness and injection speed of the raw material powder, an anchor layer (a region where the raw material powder cuts into the under layer) may be observed at a boundary between the substrate or the like and the AD film (the film formed according to the AD method).

Further, according to the embodiment, since an aerosol with stable concentration can be generated for a long period and supplied to the injection nozzle of the film forming unit, a good-quality AD film with stable film quality such as density (porosity) and film thickness can be formed.

### (Experiment 1)

An experiment for confirming the effect by the assist gas was made using the aerosol generating apparatus according to the first embodiment of the present invention.

(i) Experimental Procedure and Evaluation Method

An aerosol was generated under the following condition and collected, and the amount of the powder contained therein was measured according to the following method. That is, one end of a PFA tube (inner diameter: 3 mm, outer diameter: 4 mm) manufactured by NICHIAS Corporation was connected to the aerosol lead-out part 18 shown in FIGS. 1A and 1B, and the other end of the PFA tube was inserted into a collection bottle of 100 cc filled with water. The powder was collected in the bottle by blowing the aerosol into the water. The collection bottle was replaced every five minutes during the generation of aerosol, and the powder is obtained for 60 minutes (i.e., for 12 collection bottles). After the powder was collected, the water mixed with the powder is evaporated by a hot plate. The supplied amounts of powder were obtained by measuring the weights of the collection bottles before and after the experiment, and averaged as an average supplied amount of powder. Further, the fluctuation ranges of average supplied amounts of powder were calculated using the following equations (1) and (2), and values with larger absolute values were adopted as results of the experiment.

(fluctuation range of supplied amount of powder)={(minimum supplied amount)−(average supplied amount)}/(average supplied amount) (1)

(fluctuation range of supplied amount of powder)={(maximum supplied amount)−(average supplied amount)}/(average supplied amount) (2)

(ii) Aerosol Forming Conditions

Sample: PZT of 0.7 μm in average particle diameter

Kind of dispersion gas: pure oxygen (G2 grade)

Kind of assist gas: pure oxygen (G2 grade)

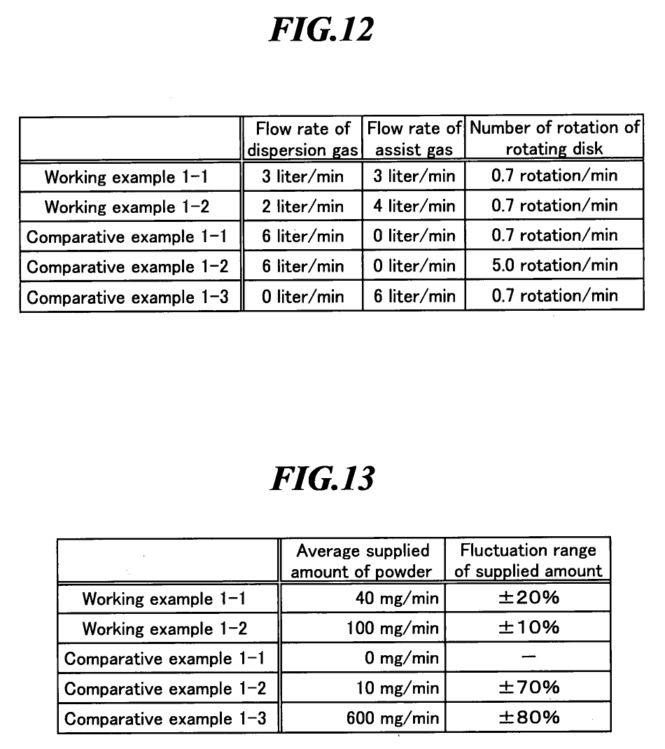

In Experiment 1, conditions of the flow rate of dispersion gas, the flow rate of assist gas, and the number of rotation of the rotating disk with respect to plural working examples and comparative examples are shown in FIG. 12. Through these working examples and comparative examples, the total amount of the flow rate of dispersion gas and the flow rate of assist gas are kept equal. Further, the distance L_{GAP} between the agitating blades and the rotating disk is set to about 0.3 mm.

(iii) Results

Results of the average supplied amounts of powder and fluctuation ranges of supplied amounts of powder in Experiment 1 are shown in FIG. 13. As shown in the results, in Comparative Example 1-1, the powder could be never supplied because the assist gas is not introduced. Further, in Comparative Example 1-2, an attempt to supply powder was made by raising the rotational speed of the rotating disk higher than that in Comparative Example 1-1, however, only a slight amount of powder (about 10 mg/min) could be supplied. Further, the fluctuation range of supplied amount was very large as ±70%. Generally, the supplied amount of powder per unit time (g/min) is determined by a product of the volume of the groove (cc), the bulk density of the powder (g/cc), and the rotational speed of the rotating disk (rotation/circumference). Furthermore, in Comparative Example 1-3, only the assist gas was introduced, however, a large amount of powder was supplied (about 600 mg/min) and the fluctuation range was very large (±80%). It is conceivable that a large amount of gas flew into the groove of the rotating disk because the amount of assist gas was too large, and the powder was carried by the airflow and went out of control.

On the other hand, in Working Example 1-1 using both the dispersion gas and the assist gas, the powder could be supplied (about 40 mg/min), and the fluctuation range fell within a small range (±20%). Further, in Working Example 1-2, the ratio of flow rates of dispersion gas and assist gas was changed, and the fluctuation range could be made smaller (±10%) Furthermore, it has been confirmed that the supplied amount of powder can be controlled by adjusting the flow rate of assist gas.

### (Experiment 2)

An experiment for confirming the effect of specifying the gap L_{GAP} between the height of the agitating blades 11 and the top 14a of the rotating disk 14 shown in FIGS. 1A and 1B was made by using the aerosol generating apparatus according to the first embodiment of the present invention.

(i) Experimental Procedure and Evaluation Method

In the same experimental procedure as that of Experiment 1, the height of the agitating blades 11 and the height of the rotating disk 14 shown in FIGS. 1A and 1B were changed. The heights were measured using a depth micrometer DMS manufactured by Mitutoyo Corporation. Further, the evaluation method was the same as that of Experiment 1.

(ii) Aerosol Forming Conditions

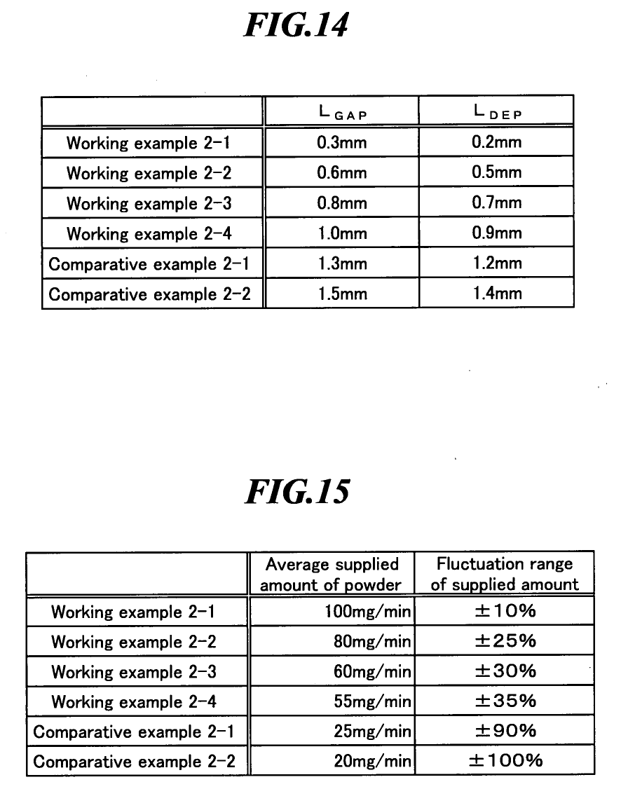

The gap L_{GAP} and the distance L_{DEP} between the inner bottom surface 1b of the powder containing chamber 1 and the top 14a of the rotating disk 14 (see FIG. 2) were set as shown in FIG. 14. Other conditions were the same as those in Working Example 1-2 of Experiment 1.

In the working examples and comparative examples, every distance (L_{GAP}−L_{DEP}) between the agitating blades 11 and the inner bottom surface 1b of the powder containing chamber 1 is 0.1 mm.

(iii) Results

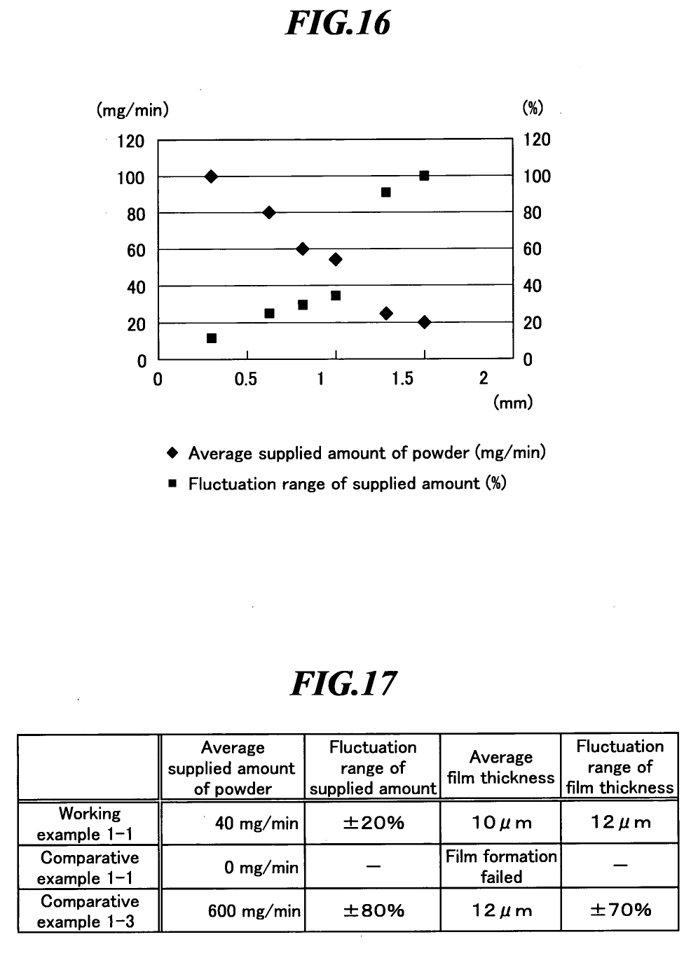

Results of the average supplied amounts of powder and fluctuation ranges of supplied amounts of powder in Experiment 2 are shown in FIGS. 15 and 16. In FIG. 16, the horizontal axis indicates the gap L_{GAP} (mm), the vertical axis on the left shows the average supplied amount of powder (mg/min), and the vertical axis on the right shows the absolute value (%) of the fluctuation range of supplied amount of powder.

As shown in FIGS. 15 and 16, in Comparative Examples 2-1 and 2-2, the fluctuation range of supplied amount of powder sharply rose (e.g., 90% for L_{GAP}=1.3 mm), and it has been found that the supplied amount of powder could not be controlled. On the other hand, in Working Examples, the fluctuation range of supplied amount of powder was near 35% at the highest (e.g., for L_{GAP}=1 mm). From the results, it has been confirmed that the supplied amount of powder becomes stable by setting L_{GAP} to 1 mm or less).

### (Experiment 3)

An experiment for forming films using the aerosol generated under the conditions of the above-mentioned working Example 1 and Comparative Example 1 was made in the film forming apparatus according to the one embodiment of the present invention.

(i) Experimental Procedure and Evaluation Method

In order to finally form a film of about 10 μm, the number of reciprocation of the substrate stage was set based on the average supplied amounts of powder (i.e., aerosol concentration). Then, in the film forming apparatus shown in FIG. 11, a film was formed by reciprocating the substrate stage at the set number of times. Such film formation was performed at six times for the respective conditions of Working Example 1-1 and Comparative Examples 1-1 and 1-3 of Experiment 1, and the film thicknesses were measured and average values were obtained. Further, the fluctuation ranges of film thicknesses were calculated using the following equations (3) and (4), and values with larger absolute values were adopted as results of the experiment.

(fluctuation range of film thickness)={(minimum film thickness)−(average film thickness)}/(average film thickness) (3)

(fluctuation range of film thickness)={(maximum film thickness)−(average film thickness)}/(average film thickness) (4)

(ii) Aerosol Forming Conditions

Substrate material: YSZ (yttria-stabilized zirconia)

Film forming temperature: room temperature

The distance L_{GAP} between the agitating blades and the rotating disk is set to about 0.3 mm.

(iii) Results

Results of the average supplied amounts of powder and fluctuation ranges of supplied amounts, and average film thicknesses and fluctuation ranges of film thicknesses in Experiment 3 are shown in FIG. 17. As shown in FIG. 17, when the aerosol of Comparative Example 1-1 was used, the powder could be never supplied and no film was formed. Further, when the aerosol of Comparative Example 1-3 was used, the fluctuation range of supplied amount of powder was very large (±80%) and the fluctuation range of film thickness was also large (±70%), and the average film thickness was largely apart from the target value 10 μm (about 12 μm).

On the other hand, when the aerosol of Working Example 1-1 was used, the film thickness near the target value (10 μm) could be obtained and the fluctuation range of film thickness could be suppressed to be small (±20%).

From the above experimental results, by using the assist gas in addition to the dispersion gas in the aerosol generating apparatus, the powder can be stably supplied to the film forming unit for a long period (e.g., 60 minutes). Further, it has been confirmed that the amount of powder can be easily controlled. Furthermore, it has been confirmed that, using thus generated aerosol, a film having a homogeneous thickness can be obtained and the control of film thickness can be taken more easily.

1. An aerosol generating apparatus comprising:
a powder containing chamber formed with an opening for leading out powder contained therein;
assisting means for assisting lead-out of the powder when the powder is led out from said opening; and
gas supplying means for supplying a gas for dispersing the powder led out from said opening.

2. The aerosol generating apparatus according to claim 1, wherein said assisting means supplies a gas into said powder containing chamber.

3. The aerosol generating apparatus according to claim 1, wherein said assisting means performs pressure regulation such that pressure within said powder containing chamber becomes higher than pressure outside of said powder containing chamber.

4. The aerosol generating apparatus according to claim 1, wherein said assisting means provides vibration and/or impact to said powder containing chamber.

5. The aerosol generating apparatus according to claim 1, wherein said assisting means pushes out the powder from said opening.

6. The aerosol generating apparatus according to claim 5, wherein said assisting means includes a piston provided within said powder containing chamber.

7. The aerosol generating apparatus according to claim 5, wherein said assisting means includes a pressing scraper provided within said powder containing chamber.

8. The aerosol generating apparatus according to claim 1, wherein said gas supplying means includes:
a tubular body formed with an opening at a position corresponding to the opening of said powder containing chamber; and
means for forming airflow within said tubular body.

9. The aerosol generating apparatus according to claim 1, further comprising:
a carrying unit, having a rotator formed with a groove having predetermined width and depth on a circle facing said opening, for carrying the powder led out from said opening and placed in the groove to a position of said gas supplying means by rotating said rotator.

10. The aerosol generating apparatus according to claim 9, wherein said assisting means includes:
rotating blades for agitating the powder contained within said powder containing chamber, said rotating blades positioned within said powder containing chamber such that a distance between said rotating blades and said rotator is not larger than 1 mm.

11. The aerosol generating apparatus according to claim 9, wherein said gas supplying means sprays the gas toward the groove formed within said rotator.

12. The aerosol generating apparatus according to claim 9, further comprising:
a path for leading out the powder carried by said carrying unit from the groove;
wherein said gas supplying means produces airflow for carrying the powder led out through said path.

13. A film forming apparatus using a film forming method of depositing raw material on a substrate by spraying an aerosol, in which powder of the raw material is dispersed in a gas, toward the substrate, said apparatus comprising:
(i) an aerosol generating apparatus including a powder containing chamber formed with an opening for leading out powder contained therein, assisting means for assisting lead-out of the powder when the powder is led out from said opening, and gas supplying means for supplying a gas for dispersing the powder led out from said opening;
(ii) a substrate stage for holding a substrate; and
(iii) a nozzle for injecting the aerosol generated by said aerosol generating apparatus toward the substrate.

14. An aerosol generating method comprising the steps of:
(a) leading out powder contained in a powder containing chamber formed with an opening from said opening while assisting lead-out of the powder; and
(b) dispersing the powder led out from said opening with a gas.

15. The aerosol generating method according to claim 14, wherein step (a) includes supplying a gas to said powder containing chamber.

16. The aerosol generating method according to claim 14, wherein step (a) includes performing pressure regulation such that pressure within said powder containing chamber becomes higher than pressure outside of said powder containing chamber.

17. The aerosol generating method according to claim 14, wherein step (a) includes providing vibration and/or impact to said powder containing chamber.

18. The aerosol generating method according to claim 14, wherein step (a) includes pushing out the powder from said powder containing chamber.

19. The aerosol generating method according to claim 18, wherein step (a) includes pushing out the powder from said powder containing chamber by using a piston.

20. The aerosol generating method according to claim 18, wherein step (a) includes pushing out the powder from said powder containing chamber by using a pressing scraper.

21. The aerosol generating method according to claim 14, wherein step (b) includes inputting the powder led out from said opening to a tubular body in which airflow is formed.

22. The aerosol generating method according to claim 14, further comprising the step of:
(a′) placing the powder led out from said opening at step (a) in a groove having predetermined width and depth and carrying the powder;
wherein step (b) includes generating an aerosol by spraying the gas to the powder carried at step (a′).

23. The aerosol generating method according to claim 14, further comprising the steps of:
(a′) placing the powder led out from said opening at step (a) in a groove having predetermined width and depth and carrying the powder; and
(a″) leading out the powder carried at step (a′) from said groove;
wherein step (b) includes producing airflow for carrying the powder led out from said groove.

24. A film forming method comprising the steps of:
(a) leading out powder of a raw material contained in a powder containing chamber formed with an opening from said opening while assisting lead-out of the powder;
(b) generating an aerosol by dispersing the powder led out from said opening with a gas; and
(c) depositing the raw material on a substrate by spraying the aerosol generated at step (b) from a nozzle toward the substrate to allow the powder to collide with the substrate.

25. A metal film formed by using the film forming method according to claim 24.

26. A ceramic film formed by using the film forming method according to claim 24.
